(19) United States
(12) Patent Application Publication
(10) Pub. No.: US 2024/0242839 A1
(43) Pub. Date: Jul. 18, 2024

(54) LEFT VENTRICULAR HYPERTROPHY PREDICTION MODEL TRAINING METHOD AND DEVICE THEREOF

(71) Applicants: National Yang Ming Chiao Tung University, Hsinchu City (TW); TAIPEI VETERANS GENERAL HOSPITAL, TAIPEI CITY (TW)

(72) Inventors: Shin-Mu TSENG, Hsinchu City (TW); Yu-Feng HU, Taipei City (TW); Chih-Min LIU, Taipei City (TW)

(21) Appl. No.: 18/308,625

(22) Filed: Apr. 27, 2023

(30) Foreign Application Priority Data
Jan. 18, 2023 (TW) 112102452

(51) Int. Cl. G16H 50/30; A61B 5/35; G06N 20/00

(52) U.S. Cl. CPC G16H 50/30; A61B 5/35; G06N 20/00

(57) ABSTRACT

A prediction model training method includes the following steps. A first model is trained according to first electrocardiograms, wherein the first model includes a feature extraction layer, and the feature extraction layer is configured to extract features corresponding to an electrocardiogram. First feature information corresponding to second electrocardiograms is extracted according to the second electrocardiograms and the feature extraction layer of the first model. A second model is trained according to the first feature information, gender information corresponding to the second electrocardiograms, and age information corresponding to the second electrocardiograms.

### CROSS-REFERENCE TO RELATED APPLICATION

This application claims priority to Taiwan Application Serial Number 112102452, filed Jan. 18, 2023, which is herein incorporated by reference in its entirety.

### BACKGROUND

### Field of Invention

The present disclosure relates to a prediction model training method and device thereof. More particularly, the present disclosure relates to a left ventricular hypertrophy prediction model training method and device thereof.

### Description of Related Art

The symptom of left ventricular hypertrophy may increase the risk of heart failure, arrhythmias, cardiovascular death, and sudden cardiac death (SCD). Due to their youthfulness, young and middle-aged patients with left ventricular hypertrophy tend to ignore the diagnosis of left ventricular hypertrophy and causes family breakdown, medical burden, and significant loss to the country.

In view of this, how to predict the risk of left ventricular hypertrophy is the goal that the industry strives to work on.

### SUMMARY

The disclosure provides a prediction model training method. The prediction model training method comprises: training a first model according to a plurality of first electrocardiograms, wherein the first model comprises a feature extraction layer, and the feature extraction layer is configured to extract feature information corresponding to electrocardiograms; extracting first feature information corresponding to a plurality of second electrocardiograms according to the plurality of second electrocardiograms and the feature extraction layer of the first model, wherein each of the plurality of second electrocardiograms is corresponding to at least one of the first feature information; and training a second model according to the first feature information, gender information corresponding to the plurality of second electrocardiograms, and age information corresponding to the plurality of second electrocardiograms.

The disclosure also provides a prediction model training device. The prediction model training device comprises a storage and a processor. The storage is configured to store a first model and a second model. The processor couples to the storage. The processor is configured to train the first model according to a plurality of first electrocardiograms, wherein the first model comprises a feature extraction layer, and the feature extraction layer is configured to extract a plurality of features corresponding to an electrocardiogram. The processor is further configured to extract first feature information corresponding to a plurality of second electrocardiograms according to the plurality of second electrocardiograms and the feature extraction layer of the first model, wherein each of the plurality of second electrocardiograms is corresponding to at least one of the first feature information. The processor is further configured to train the second model according to the first feature information, gender information corresponding to the plurality of second electrocardiograms, and age information corresponding to the plurality of second electrocardiograms.

The disclosure also provides a prediction model training device. The prediction model training device comprises a storage and a processor. The storage is configured to store a first model and a second model. The processor couples to the storage. The processor is configured to train a first model according to a plurality of first electrocardiograms, wherein the first model comprises a feature extraction layer, and the feature extraction layer is configured to extract a plurality of features corresponding to an electrocardiogram. The processor is further configured to extract first feature information corresponding to a plurality of second electrocardiograms according to the plurality of second electrocardiograms and the feature extraction layer of the first model, wherein each of the plurality of second electrocardiograms is corresponding to at least one of the first feature information. The processor is further configured to train a second model according to the first feature information, gender information corresponding to the plurality of second electrocardiograms, and age information corresponding to the plurality of second electrocardiograms, wherein the second model is configured to generate a prediction result according to the feature information generated by the first model, the gender information corresponding to the electrocardiogram, and the age information corresponding to the electrocardiogram, and the prediction result is configured to indicate whether a patient corresponding to the electrocardiogram has a symptom of left ventricular hypertrophy.

It is to be understood that both the foregoing general description and the following detailed description are by examples, and are intended to provide further explanation of the disclosure as claimed.

### BRIEF DESCRIPTION OF THE DRAWINGS

The disclosure can be more fully understood by reading the following detailed description of the embodiment, with reference made to the accompanying drawings as follows:

FIG. 1 is a schematic diagram illustrating a prediction model training device according to some embodiments of the present disclosure.

FIG. 2 is a flow diagram illustrating a prediction model training method according to some embodiments of the present disclosure.

FIG. 3 is a flow diagram illustrating a first model training method according to some embodiments of the present disclosure.

FIG. 4A is a flow diagram illustrating calculations of a first submodel according to some embodiments of the present disclosure.

FIG. 4B is a flow diagram illustrating calculations of a second submodel according to some embodiments of the present disclosure.

FIG. 4C is a flow diagram illustrating calculations of a third submodel according to some embodiments of the present disclosure.

FIG. 4D is a flow diagram illustrating calculations of a fourth submodel according to some embodiments of the present disclosure.

FIG. 4E is a flow diagram illustrating calculations of a fifth submodel according to some embodiments of the present disclosure.

FIG. 5 is a flow diagram illustrating a feature information extraction method according to some embodiments of the present disclosure.

FIG. 6 is a flow diagram illustrating a second model training method according to some embodiments of the present disclosure.

FIG. 7A is a flow diagram illustrating calculations of a sixth submodel according to some embodiments of the present disclosure.

FIG. 7B is a flow diagram illustrating calculations of a seventh submodel according to some embodiments of the present disclosure.

### DETAILED DESCRIPTION

Reference will now be made in detail to the present embodiments of the disclosure, examples of which are illustrated in the accompanying drawings. Wherever possible, the same reference numbers are used in the drawings and the description to refer to the same or like parts.

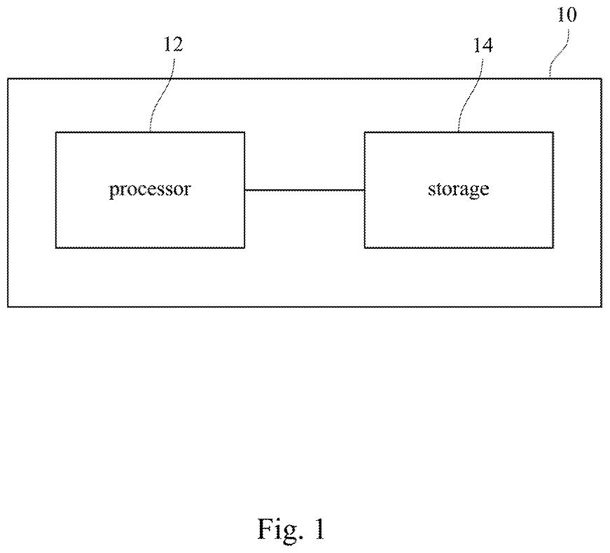

Reference is made to FIG. 1. FIG. 1 is a schematic diagram illustrating a prediction model training device 10 according to some embodiments of the present disclosure. The prediction model training device 10 is configured to train a prediction model, and the prediction model is configured to predict the risk of patients suffering from symptoms of left ventricular hypertrophy according to electrocardiograms (ECGs). The prediction model training device 10 comprises a processor 12 and a storage 14.

The storage 14 is configured to store electrocardiograms, wherein the electrocardiograms comprise a label indicating whether the patients corresponding to the electrocardiograms have symptoms of left ventricular hypertrophy. In some embodiments, the electrocardiograms are the record of clinical patients measured in the hospital, and the clinical patients have also undergone cardiac ultrasound examinations. After the doctor judges whether the clinical patients have the symptoms of left ventricular hypertrophy, the electrocardiograms are then marked. In some embodiments, the electrocardiograms comprise 10 seconds of recordings at 500 Hz for 12 leads. Namely, the electrocardiograms generated by a single measurement of a patient comprise 12 electrocardiogram waveforms with different leads and measured at the same time. The duration of each electrocardiogram waveforms is 10 seconds, and the sampling frequency is 500 times per second.

The storage 14 is further configured to store gender information and age information corresponding to the electrocardiograms. The gender information and the age information record the gender and age of patients corresponding to the electrocardiograms. In some embodiments, the age information is divided into 7 grades such as 0-20 years old, 21-30 years old, 31-40 years old, 41-50 years old, 51-60 years old, and above 60 years old.

In some embodiments, the prediction model training device 10 can also receive the electrocardiograms (e.g., first electrocardiograms and second electrocardiograms) configured to train a first model and a second model, the gender information corresponding to the electrocardiograms, and the age information corresponding to the electrocardiograms from external device (e.g., cloud database) and perform training and calculation accordingly.

The storage 14 is also configured to store the first model and the second model, wherein the first model is configured to generate feature information according to electrocardiograms, and the second model is configured to generate a prediction result according to the feature information generated by the first model, the gender information corresponding to the electrocardiograms, and the age information corresponding to the electrocardiograms. The prediction result is configured to indicate whether the patients corresponding to the electrocardiograms have the symptoms of left ventricular hypertrophy.

In some embodiments, the first model comprises a first submodel, a second submodel, a third submodel, a fourth submodel, and a fifth submodel. In some embodiments, the second model comprises the fifth submodel, a sixth submodel, and a seventh submodel.

For example, the first model can be composed by a plurality of submodels, and the submodels comprise a mapping submodel (i.e., the fourth submodel), a feature extraction submodel (i.e., the second submodel), a first dimension transformation submodel (i.e., the first submodel), a second dimension transformation submodel (i.e., the third submodel), and a probability transformation submodel (i.e., the fifth submodel).

In some embodiments, the storage 14 can comprise a semiconductor or solid-state memory, a magnetic tape, a removable computer diskette, a random-access memory (RAM), a read-only memory (ROM), a rigid magnetic disk, and/or an optical disk.

The processor 12 is configured to train the first model and the second model according to the electrocardiograms, the gender information, and the age information stored in the storage 14. Details about the training of the first model and the second model will be discussed in following paragraphs.

In some embodiments, the processor 12 can comprise a central processing unit (CPU), a graphics processing unit (GPU), a multi-processor, a distributed processing system, an application specific integrated circuit (ASIC), and/or a suitable processing unit.

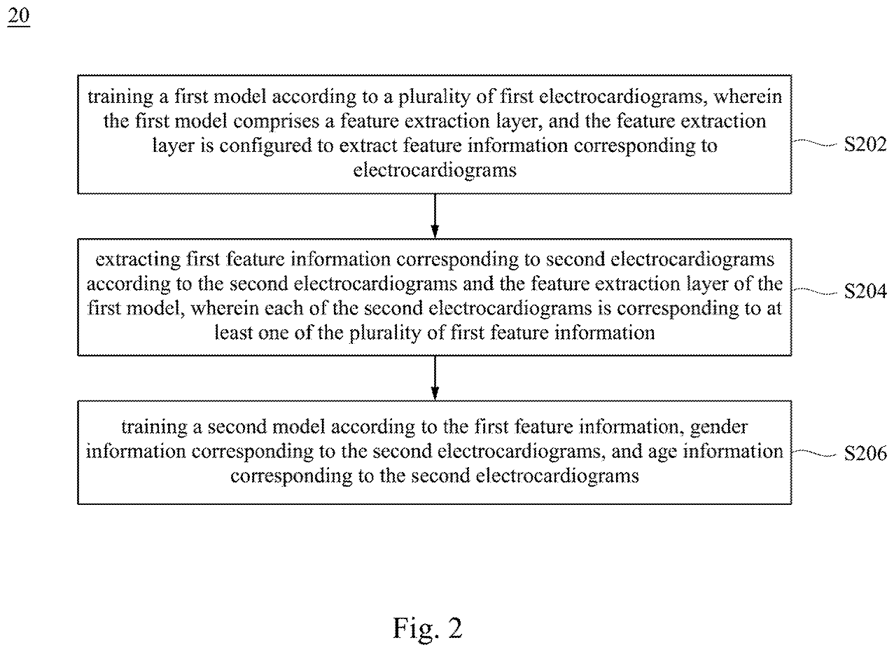

Reference is made in FIG. 2. FIG. 2 is a flow diagram illustrating a prediction model training method 20 according to some embodiments of the present disclosure. The prediction model training method 20 comprises steps S202-S206. The prediction model training method 20 is configured to train the first model and the second model according to the electrocardiograms, the gender information, and the age information, and the prediction model training method 20 can be executed by the prediction model training device 10 shown in FIG. 1.

First, in the step S202, the processor 12 of the prediction model training device 10 trains the first model according to a plurality of first electrocardiograms, wherein the first model comprises a feature extraction layer, and the feature extraction layer is configured to extract feature information corresponding to electrocardiograms.

Next, in the step S204, the processor 12 of the prediction model training device 10 extracts first feature information corresponding to a plurality of second electrocardiograms according to the plurality of second electrocardiograms and the feature extraction layer of the first model, wherein each of the plurality of second electrocardiograms is corresponding to at least one of the first feature information.

Finally, in the step S206, the processor 12 of the prediction model training device 10 trains a second model according to the first feature information, gender information corresponding to the plurality of second electrocardiograms, and age information corresponding to the plurality of second electrocardiograms.

In some embodiments, the second electrocardiograms and the first electrocardiograms can be the same training data set to enhance the feature extraction ability of the feature extraction layer of the first model and then improve the accuracy of the second model.

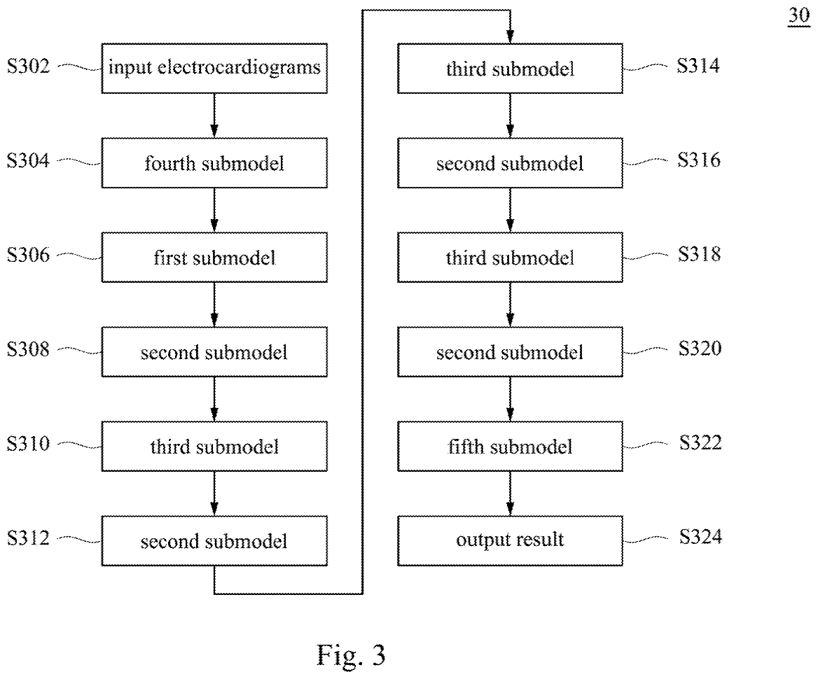

Details about the training of the first model are shown in FIG. 3. FIG. 3 is a flow diagram illustrating a first model training method 30 according to some embodiments of the present disclosure. The first model training method 30 comprises steps S302-S324, and the first model training method 30 is configured to adjust the parameters of the first model according to electrocardiograms to train the first model. The first model training method 30 can be executed by the prediction model training device 10 shown in FIG. 1. The first model training method 30 comprises performing calculation by using the first submodel, the second submodel, the third submodel, the fourth submodel, and the fifth submodel according to a specific order and forming the first model, and each of the submodels mentioned above comprises a plurality of layers. In some embodiments, the first model training method 30 trains the first model based on a first composition order, wherein the first composition order corresponds to the fourth submodel, the first submodel, the second submodel, the third submodel, the second submodel, the third submodel, the second submodel, the third submodel, the second submodel, and the fifth submodel to form the first model.

Furthermore, the first model comprises a feature extraction layer configured to extract the feature information of electrocardiograms. In this embodiment, the feature extraction layer comprises each of the submodels in the steps S304-S320. Namely, after electrocardiograms are calculated by using each of the submodels in the steps S304-S320 (i.e., the feature extraction layer) in the first model training method 30, the feature information corresponding to the electrocardiograms can be extracted.

It is noticed that, the plurality of second submodels or the plurality of third submodels of the first model only represent the layer structures of the second submodels or the third submodels are the same, yet the parameters of the second submodels or the third submodels may be different after adjustments with different orders.

First, in the step S302, the processor 12 of the prediction model training device 10 obtains a plurality of input electrocardiograms. In some embodiments, the input electrocardiograms are the first electrocardiograms mentioned above.

In some embodiments, the processor 12 edits electrocardiograms into a plurality of electrocardiogram segments according to a plurality of heartbeat record in the electrocardiograms and takes the electrocardiogram segments as the first electrocardiograms mentioned above, wherein each of the electrocardiogram segments corresponds to a time interval in the electrocardiograms.

In some embodiments, the input electrocardiograms are electrocardiograms comprising 10 seconds of recordings mentioned above. The processor 12 selects 8 heartbeat recordings randomly from the heartbeat recordings in the electrocardiograms, edits the 8 heartbeat recordings into 8 electrocardiogram segments, and takes the 8 electrocardiogram segments as the input electrocardiograms respectively.

In some embodiments, each of first electrocardiogram segments comprises a time interval corresponding to a peak, and each of second electrocardiogram segments also comprises a time interval corresponding to a peak. For example, each of the first electrocardiogram segments can be an electrocardiogram segment from 0.2 seconds before an R wave peak to 0.4 seconds after the R wave peak. It is noticed that, the time interval can be a specific length of time or a time frame between two specific point of time in an electrocardiogram.

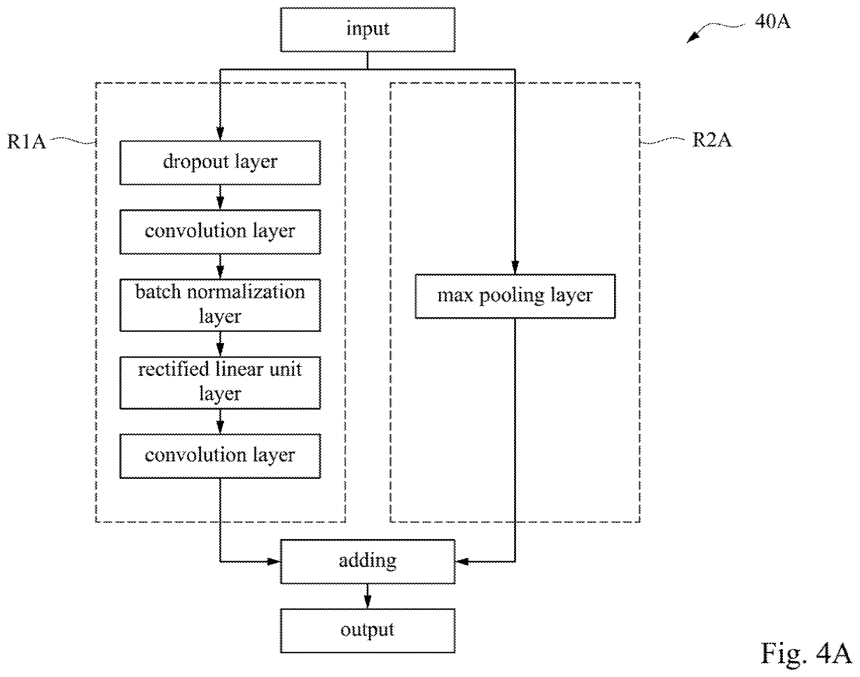

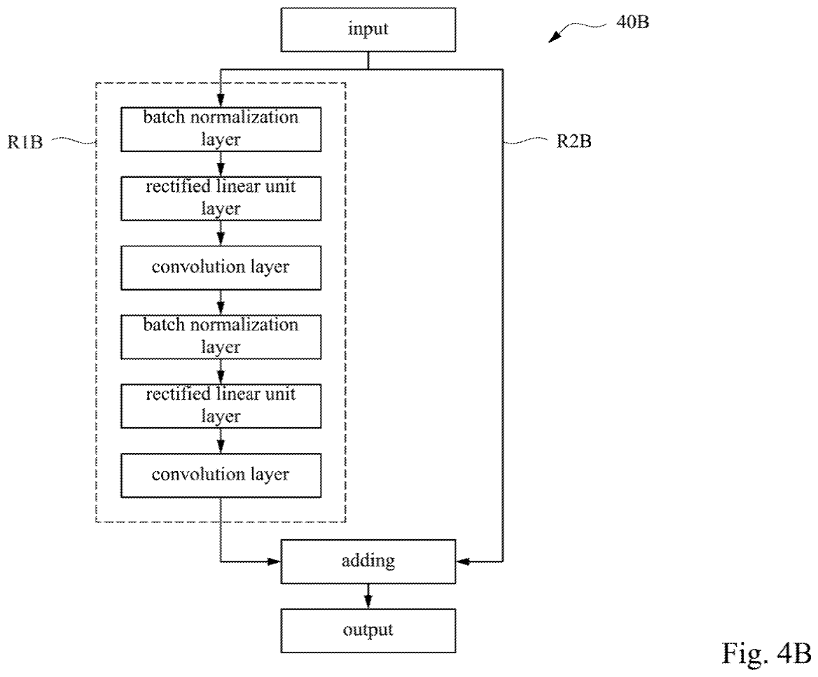

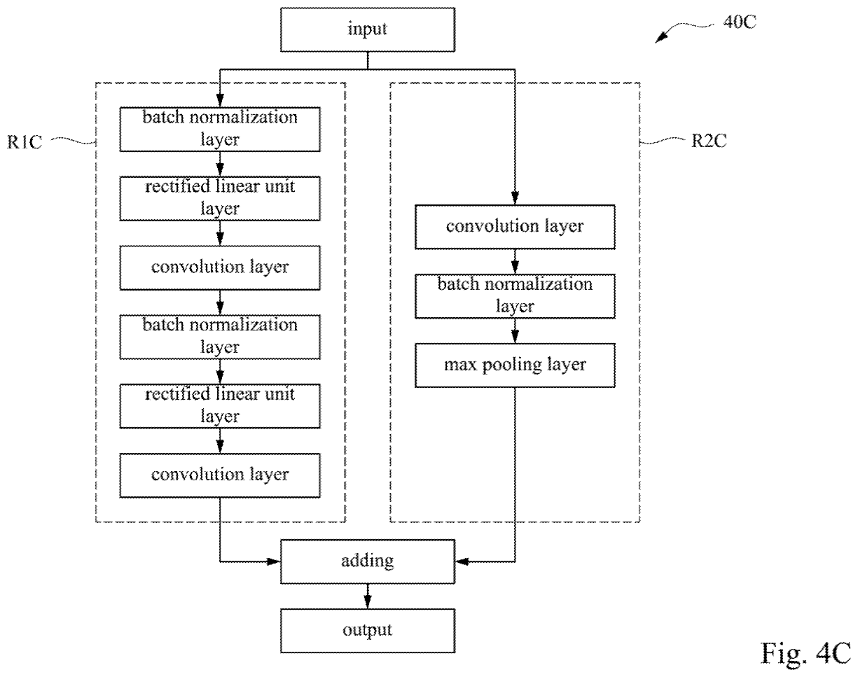

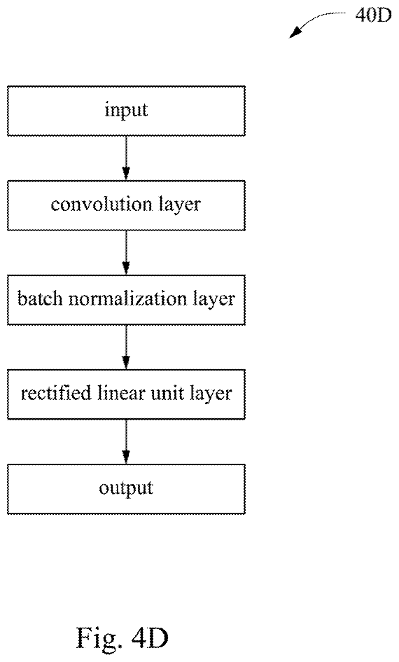

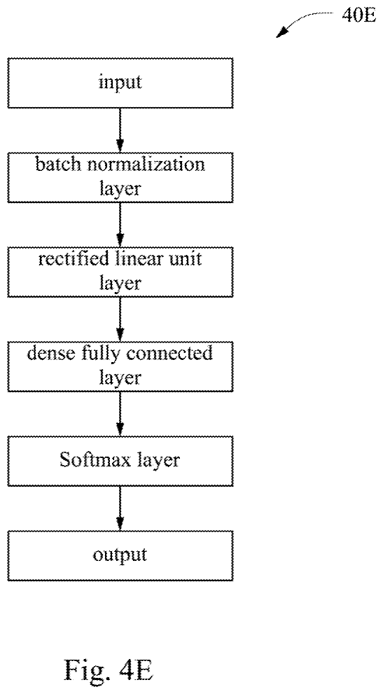

Next, in the step S304, the processor 12 performs calculations according to the input electrocardiograms by using the fourth submodel, wherein the fourth submodel is configured to transform input electrocardiograms and map the input electrocardiogram into vectors. The layers in the fourth submodel are shown in FIG. 4D. As shown in FIG. 4D, the fourth submodel 40D comprises a convolution layer, a batch normalization (BN) layer, and a rectified linear unit (ReLU) layer in sequence.

The convolution layer is configured to transform input signals (e.g., electrocardiograms or vectors) and map the input signals into vectors. Specifically, the signals are calculated by using masks to achieve filtering, transformation, and mapping function.

Furthermore, the output of the convolution layer is taken as the input of the batch normalization layer. The batch normalization layer is an activation function and is configured to balance parameters at different scales, and then increase the depth of a model (i.e., the first model), that is, increase the number of vectors.

Next, the output of the batch normalization layer is taken as the input of the rectified linear unit layer. The rectified linear unit is an activation function and is configured to strengthen the nonlinear characteristics of a model.

Finally, the output of the rectified linear unit layer is the output of the fourth submodel 40D. In some embodiments, the number of dimensions of the output signal of the fourth submodel 40D in the step S304 is 64.

Next, in the step S306, the processor 12 takes the output of the fourth submodel 40D in the step S304 as an input signal and performs calculations by using the first model, wherein the first model is configured to extract features from the signal by using the combination of the convolution layers. Additionally, the processor 12 also performs dimension number transformation by using skip connection to solve the problem of poor training result with deep model depth.

The multiple layers of the first submodel are shown in FIG. 4A. As shown in FIG. 4A, a first submodel 40A comprises calculation paths R1A and R2A, and the calculation path R1A comprises a dropout layer, a convolution layer, a batch normalization layer, a rectified linear unit layer, and a convolution layer in sequence. Additionally, the first submodel 40A also comprises a shortcut connection (also called shortcut) (i.e., the calculation path R2A). The calculation path R2A comprises a max pooling layer.

The dropout layer is configured to discard part of the parameter weights to prevent models form overfitting, and the max pooling layer is configured to compress the input signal to transform the dimension of the input signal while preserving important features. Since the structures and the functions of the convolution layers, the batch normalization layer, and the rectified linear unit layer are the same as the structures and the functions of the convolution layer, the batch normalization layer, and the rectified linear unit layer shown in the fourth submodel 40D in FIG. 4D, details of those layers in the first submodel 40A have been simplified for the purposes of clarity.

In the calculations of the first submodel 40A, the input signal passes through the layers in the calculation paths R1A and R2A and is calculated separately, and output signals are generated accordingly. Next, as shown in the drawing, the output of the first submodel 40A is calculated by adding the two outputs of the calculation paths R1A and R2A. In some embodiments, the number of dimensions of the output signal of the first submodel 40A in the step S306 is 64.

Next, in the step S308, the processor 12 takes the output of the first submodel 40A in the step S306 as an input signal and performs calculations by using the second model, wherein the first model is configured to extract features from the signal by using the combination of the convolution layers. Additionally, the processor 12 also performs dimension number transformation by using skip connection to solve the problem of poor training result with deep model depth.

The multiple layers of the second submodel are shown in FIG. 4B. As shown in FIG. 4B, a second submodel 40B comprises calculation paths R1B and R2B, and the calculation path R1B comprises a batch normalization layer, a rectified linear unit layer, a batch normalization layer, a rectified linear unit layer, and a convolution layer in sequence. Additionally, the second submodel 40B also comprises a shortcut connection (i.e., the calculation path R2B). Since the structures and the functions of the convolution layers, the batch normalization layer, and the rectified linear unit layer are the same as the structures and the functions of the convolution layer, the batch normalization layer, and the rectified linear unit layer shown in the fourth submodel 40D in FIG. 4D, details of those layers in the second submodel 40B have been simplified for the purposes of clarity.

In the calculations of the second submodel 40B, the input signal passes through the layers in the calculation paths R1B, and output signals are generated accordingly, wherein the output signal of the calculation paths R2B is the input signal. Next, as shown in the drawing, the output of the second submodel 40B is calculated by adding the two outputs of the calculation paths R1B and R2B. In some embodiments, the number of dimensions of the output signal of the second submodel 40B in the step S308 is 64.

Furthermore, in the step S310, the processor 12 takes the output of the second submodel 40B in the step S308 as an input signal and performs calculations by using the third model, wherein the third model is configured to extract features from the signal by using the combination of the convolution layers. Additionally, the processor 12 also performs dimension number transformation by using skip connection to solve the problem of poor training result with deep model depth.

The multiple layers of the third submodel are shown in FIG. 4C. As shown in FIG. 4C, a third submodel 40C comprises calculation paths R1C and R2C, and the calculation path R1C comprises a batch normalization layer, a rectified linear unit layer, a batch normalization layer, a rectified linear unit layer, and a convolution layer in sequence. Additionally, the third submodel 40C also comprises a shortcut connection (i.e., the calculation path R2C). The calculation path R2C comprises a convolution layer, a batch normalization layer, and a max pooling layer in sequence. Since the structures and the functions of the convolution layers, the batch normalization layer, the rectified linear unit layer, and the max pooling layer are the same as the structures and the functions of the convolution layers, the batch normalization layer, and the rectified linear unit layer shown in the fourth submodel 40D in FIG. 4D and the max pooling layer shown in the first submodel 40A in FIG. 4A, details of those layers in the third submodel 40C have been simplified for the purposes of clarity.

In the calculations of the third submodel 40C, the input signal passes through the layers in the calculation paths R1C and R2C and is calculated separately, and output signals are generated accordingly. Next, as shown in the drawing, the output of the third submodel 40C is calculated by adding the two outputs of the calculation paths R1C and R2C. In some embodiments, the number of dimensions of the output signal of the third submodel 40C in the step S310 is 128.

Next, in the steps S312 to S320, the processor 12 performs calculations by using the second submodel 40B and the third submodel 40C respectively.

As shown in FIG. 3, in the step S312, the processor 12 takes the output of the third submodel 40C in the step S310 as an input signal and performs calculations by using the second submodel 40B; in the step S314, the processor 12 takes the output of the second submodel 40B in the step S312 as an input signal and performs calculations by using the third submodel 40C; in the step S316, the processor 12 takes the output of the third submodel 40C in the step S314 as an input signal and performs calculations by using the second submodel 40B; in the step S318, the processor 12 takes the output of the second submodel 40B in the step S316 as an input signal and performs calculations by using the third submodel 40C; and in the step S320, the processor 12 takes the output of the third submodel 40C in the step S318 as an input signal and performs calculations by using the second submodel 40B.

In some embodiments, the number of dimensions of the output signal of the second submodel 40B in the step S312 is 128; the number of dimensions of the output signal of the third submodel 40C in the step S314 is 256; the number of dimensions of the output signal of the second submodel 40B in the step S316 is 256; the number of dimensions of the output signal of the third submodel 40C in the step S318 is 512; and the number of dimensions of the output signal of the second submodel 40B in the step S320 is 512.

Finally, in the step S322, the processor 12 takes the output of the second submodel 40B in the step S320 as an input signal and performs calculations by using the fifth model, wherein the fifth model is configured to transform the input signal into a probability distribution by using Softmax function. The multiple layers of the fifth submodel are shown in FIG. 4E. As shown in FIG. 4E, a fifth submodel 40E comprises a batch normalization layer, a rectified linear unit layer, a dense fully connected layer, and a Softmax layer in sequence.

The dense fully connected layer is configured to map and transform the input signal, and the Softmax layer is configured to transform the input signal into a probability distribution. In some embodiments, the number of dimensions of the output signal of the Softmax layer is 2, that is, the output signal indicates the possibility of prediction result is positive or negative. In this embodiment, the output signal of the fifth submodel 40E in the step S322 is configured to indicate the positive and negative possibility of predicting the input electrocardiograms corresponding to symptoms of left ventricular hypertrophy. Since the structures and the functions of the batch normalization layer and the rectified linear unit layer are the same as the structures and the functions of the batch normalization layer and the rectified linear unit layer shown in the fourth submodel 40D in FIG. 4D, details of those layers in the fifth submodel 40E have been simplified for the purposes of clarity.

As shown in the drawing, in the step S322, the output of the fifth submodel 40E is the output of the Softmax layer of the fifth submodel 40E. Furthermore, as shown in the step S324 in FIG. 3, the output of the first model is the output of the fifth submodel 40E, and the output of the first model is configured to indicate whether the patients corresponding to the electrocardiograms have the symptoms of left ventricular hypertrophy. In some embodiments, the number of dimensions of the output signal of the fifth submodel 40E in the step S322 is 64.

In summary, after the calculation of the processor 12, the first model training method 30 can generate the prediction result according to the electrocardiograms. Furthermore, the first model training method 30 can validate the prediction result according to the label of the electrocardiograms, adjust the parameters of the first model, and perform the operations of calculation, prediction result generating, validation, and adjustment to obtain the trained first model.

Reference is made to FIG. 2, in the step S204 of the prediction model training method 20, the processor 12 of the prediction model training device extracts the first feature information corresponding to the second electrocardiograms according to the second electrocardiograms and the feature extraction layer of the first model, wherein each of the second electrocardiograms is corresponding to at least one of the first feature information. Details about the feature information extraction will be discussed in following paragraphs.

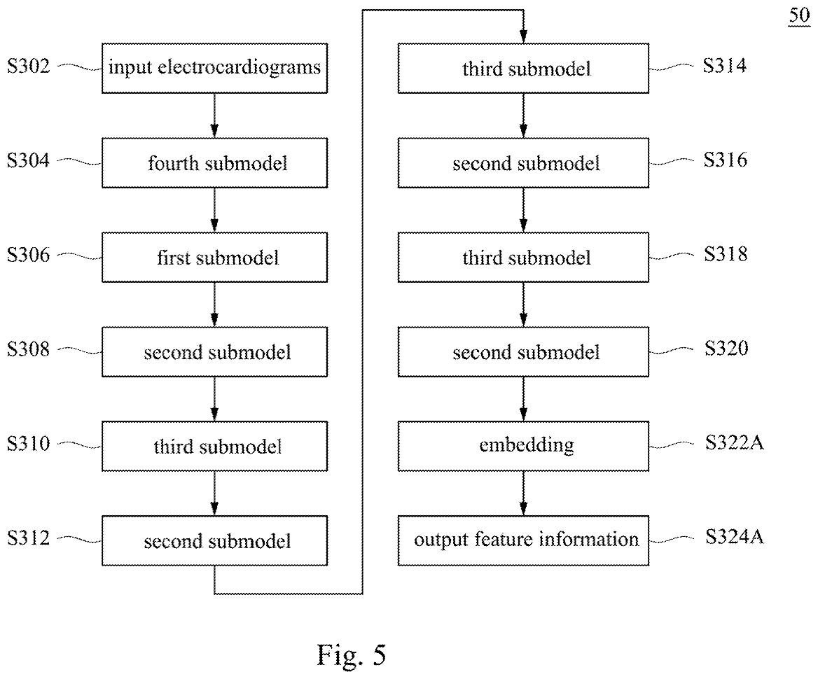

Reference is made to FIG. 3 and FIG. 5. FIG. 5 is a flow diagram illustrating a feature information extraction method 50 according to some embodiments of the present disclosure. The feature information extraction method 50 comprises steps S302-S320, step S322A, and step S324A and is configured to extract feature information according to the second electrocardiograms by using the trained first model. The feature information extraction method 50 can be executed by the prediction model training device 10 shown in FIG. 1. As shown in FIG. 3 and FIG. 5, the steps S302-S320 of the feature information extraction method 50 are the same as the steps S302-S320 of the first model training method 30. For clarity, the following paragraph will describe steps different from those in the first model training method 30 of the feature information extraction method 50.

Additionally, the step S322A of the feature information extraction method 50 inputs the output signal of the second submodel 40B in the step S320 into an embedding layer, wherein the embedding layer is configured to decrease the dimensions of the input signal and output feature information with lower dimensions. Next, in the step S324A, the feature information extraction method 50 outputs the feature information provided from the embedding layer. In some embodiments, the embedding layer maps the output signal of the second submodel 40B with 512 dimensions into the feature information with a length of 9216.

In summary, the step S204 of the prediction model training method 20 can extract feature information according to the second electrocardiograms by using the feature extraction layer of the trained first model through the feature information extraction method 50. In some embodiments, the second electrocardiograms are the first electrocardiograms. In some embodiments, the second electrocardiograms are at least one electrocardiogram stored in the storage 14, wherein the at least one electrocardiogram does not belong to the first electrocardiograms.

In some embodiments, the step S204 performs calculation of the second electrocardiograms by using the first model trained by the first electrocardiograms, thus, the specifications of the second electrocardiograms are the same as the first electrocardiograms, e.g., electrocardiogram segments from 0.2 seconds before an R wave peak to 0.4 seconds after the R wave peak.

Reference is made to FIG. 2, in the step S206 of the prediction model training method 20, the processor 12 of the prediction model training device 10 trains the second model according to the first feature information, gender information corresponding to the plurality of second electrocardiograms, and age information corresponding to the plurality of second electrocardiograms. Details about the training of the second model will be discussed in following paragraphs.

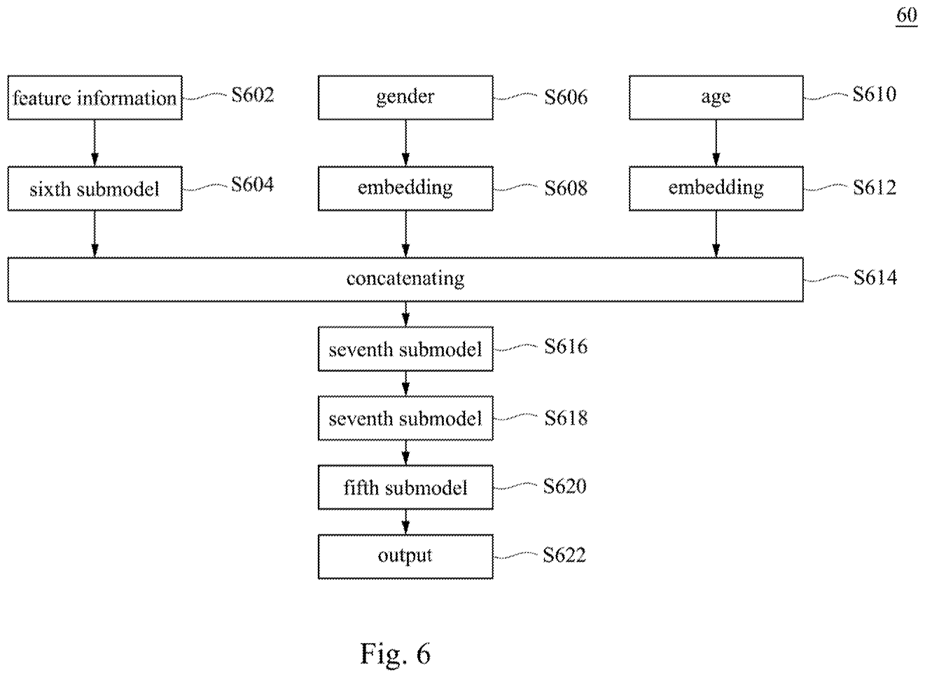

Reference is further made to FIG. 6. FIG. 6 is a flow diagram illustrating a second model training method 60 according to some embodiments of the present disclosure. The second model training method 60 comprises steps S602-S622 and is configured to train the second model according to the feature information, the corresponding gender information, and the corresponding age information. The second model training method 60 can be executed by the prediction model training device 10 shown in FIG. 1.

First, in the step S602, the processor 12 of the prediction model training device 10 obtains feature information. In some embodiments, the feature information is feature information generated by the first model.

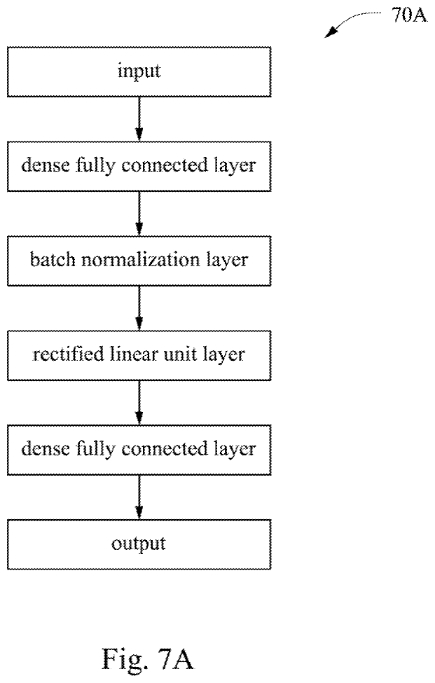

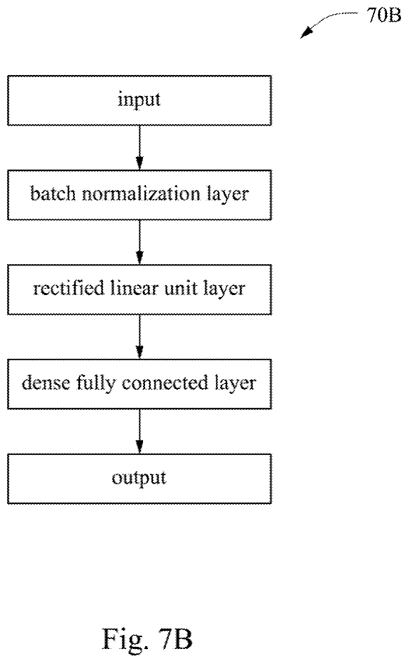

Furthermore, in the step S604, the processor 12 performs calculation according to the feature information by using a sixth model, wherein the layers of the sixth model are shown in FIG. 7A. As shown in FIG. 7A, the sixth submodel 70A comprises a dense fully connected layer, a batch normalization layer, a rectified linear unit layer, and a dense fully connected layer in sequence. Since the structures and the functions of the batch normalization layer, the rectified linear unit layer, and the dense fully connected layer are the same as the structures and the functions of the batch normalization layer and the rectified linear unit layer shown in the fourth submodel 40D in FIG. 4D and the dense fully connected layer shown in the fifth submodel 40E in FIG. 4E, details of those layers in the sixth submodel 70A have been simplified for the purposes of clarity. In some embodiments, after the feature information is calculated by using the first dense fully connected layer, the output signal is generated with 1024 dimensions; and after the output signal of the rectified linear unit layer is calculated by using the second dense fully connected layer, the output signal is generated with 256 dimensions.

In some embodiments, the feature information obtained in the step S602 comprises feature information generated from the electrocardiograms corresponding to a measurement taken from the patient. As the embodiment mentioned above, in response to the second electrocardiograms are electrocardiogram segments (e.g., 8 electrocardiogram segments) edited from electrocardiograms, the processor 12 takes the feature information generated according to the electrocardiogram segments edited from the same electrocardiograms (e.g., 8 feature information generated from the 8 electrocardiogram segments) by using the first model in the step S602 as the input signal of the sixth submodel 70A in the step S604.

On the other hand, in the step S606, the processor 12 obtains the gender information, wherein the gender information is configured to indicate the biological sex of the patient of the second electrocardiograms corresponding to the feature information. In some embodiments, the gender information is represented by 2 bits.

Next, in the step S608, the processor 12 inputs the gender information into the embedding layer and maps the gender information into a vector. In some embodiments, the gender information is mapped into a vector with 64 dimensions.

On the other hand, in the step S610, the processor 12 obtains the age information, wherein the age information is configured to indicate the age of the patient of the second electrocardiograms corresponding to the feature information. In some embodiments, the age information comprises 7 bits to indicate 7 grades.

Next, in the step S612, the processor 12 inputs the age information into the embedding layer and maps the age information into a vector. In some embodiments, the age information is mapped into a vector with 64 dimensions.

Next, in the step S614, the processor 12 concatenates the vectors outputted in the steps S604, S608, and S612. In some embodiments, the step S614 outputs a vector with 384 dimensions after the vector concatenation.

After that, in the step S616, the processor 12 performs calculation according to the vector outputted in the step S614 by using the seventh submodel, wherein the layers of the seventh submodel are shown in FIG. 7B. As shown in FIG. 7B, a seventh submodel 70B comprises a batch normalization layer, a rectified linear unit layer, and a dense fully connected layer in sequence. Since the structures and the functions of the batch normalization layer, the rectified linear unit layer, and the dense fully connected layer are the same as the structures and the functions of the batch normalization layer and the rectified linear unit layer shown in the fourth submodel 40D in FIG. 4D and the dense fully connected layer shown in the fifth submodel 40E in FIG. 4E, details of those layers in the seventh submodel 70B have been simplified for the purposes of clarity. In some embodiments, the number of the dimensions of the output signal of the seventh submodel 70B in the step S616 is 512.

Next, in the step S618, the processor 12 performs calculation according to the vector outputted in the step S616 by using the seventh submodel again. Since the calculation is the same as the calculation in the step S616, details of the calculation in the step S618 have been simplified for the purposes of clarity. In some embodiments, the number of the dimensions of the output signal of the seventh submodel 70B in the step S618 is 512.

Next, in the step S620, the processor 12 performs calculation according to the vector outputted in the step S618 by using the fifth submodel 40E. However, the output signal of the Softmax layer of the fifth submodel 40E in the step S620 is configured to indicate the result of predicting whether the corresponding patients have the symptoms of left ventricular hypertrophy according to the feature information, the gender information, and the age information. In some embodiments, the fifth submodel 40E in the step S618 outputs a signal with 2 dimensions, and the dimensions indicate the positive and negative possibility of the prediction result.

Finally, in the step S622, the processor 12 generates a prediction result according to the output of the step S620. Furthermore, the second model training method 60 can validate the prediction result according to the label of the electrocardiograms, adjust the parameters of the second model, and perform the operations of calculation, prediction result generating, validation, and adjustment to obtain the trained second model.

In summary, the prediction model training method 20 executed by the prediction model training device 10 can trains a first model by using electrocardiograms first, extracts feature information according to the electrocardiograms by using a feature layer of the trained first model, and trains a second model according to the feature information and gender information and age information corresponding to patients of the electrocardiograms. After the first model and the second model are trained, the user can inputs electrocardiograms into the first model to obtain feature information, inputs the feature information and gender information and age information of patients into the second model to generate a prediction result, and then predicts whether the patients have the symptoms of left ventricular hypertrophy and/or the possibility of the patients have the symptoms of left ventricular hypertrophy.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A prediction model training method, being adapted for use in an electronic apparatus, wherein the prediction model training method comprises the following steps:
training a first model according to a plurality of first electrocardiograms, wherein the first model comprises a feature extraction layer, and the feature extraction layer is configured to extract feature information corresponding to electrocardiograms;
extracting first feature information corresponding to a plurality of second electrocardiograms according to the plurality of second electrocardiograms and the feature extraction layer of the first model, wherein each of the plurality of second electrocardiograms is corresponding to at least one of the first feature information; and
training a second model according to the first feature information, gender information corresponding to the plurality of second electrocardiograms, and age information corresponding to the plurality of second electrocardiograms.

2. The prediction model training method of claim 1, further comprising:
receiving a patient electrocardiogram, patient gender information, and patient age information corresponding to a patient;
extracting patient feature information corresponding to the patient electrocardiogram based on the feature extraction layer; and
inputting the patient feature information, the patient gender information, and the patient age information into the second model to generate a prediction result;
wherein the prediction result is configured to indicate whether the patient corresponding to the patient electrocardiogram has a symptom of left ventricular hypertrophy.

3. The prediction model training method of claim 1, wherein the step of training the first model further comprises:
obtaining a plurality of first electrocardiogram segments from each of the plurality of first electrocardiograms, wherein each of the plurality of first electrocardiogram segments corresponds to a time interval in the plurality of first electrocardiograms; and
training the first model according to the plurality of first electrocardiogram segments corresponding to the plurality of first electrocardiograms.

4. The prediction model training method of claim 3, wherein the step of extracting the first feature information corresponding to the plurality of second electrocardiograms further comprises:
obtaining a plurality of second electrocardiogram segments from each of the plurality of second electrocardiograms, wherein each of the plurality of second electrocardiogram segments corresponds to the time interval in the plurality of second electrocardiograms; and
extracting the first feature information corresponding to the plurality of second electrocardiograms based on the plurality of second electrocardiogram segments.

5. The prediction model training method of claim 4, wherein each of the plurality of first electrocardiogram segments comprises the time interval corresponding to a peak, and each of the plurality of second electrocardiogram segments comprises the time interval corresponding to the peak.

6. The prediction model training method of claim 1, wherein the first model comprises a first submodel, a second submodel, a third submodel, a fourth submodel, and a fifth submodel, and the step of training the first model further comprises:
training the first model based on a first composition order;
wherein the first composition order corresponds to the fourth submodel, the first submodel, the second submodel, the third submodel, the second submodel, the third submodel, the second submodel, the third submodel, the second submodel, and the fifth submodel.

7. The prediction model training method of claim 1, wherein the second model comprises a fifth submodel, a sixth submodel, and a seventh submodel, and the step of training the second model further comprises:
generating second feature information according to the first feature information and the sixth submodel;
generating a gender vector according to the gender information;
generating an age vector according to the age information; and
training the second model based on the second feature information, the gender vector, the age vector, and a second composition order, wherein the second composition order corresponds to the seventh submodel, the seventh submodel, and the fifth submodel.

8. The prediction model training method of claim 6, wherein the first submodel is configured to perform the following steps:
generating a first output corresponding to an input based on a dropout layer, a plurality of convolution layers, a batch normalization layer, and a rectified linear unit layer;
inputting the input into a max pooling layer to generate a second output; and
adding the first output and the second output to generate a third output, and taking the third output as an output of the first submodel.

9. The prediction model training method of claim 6, wherein the second submodel is configured to perform the following steps:
generating a first output corresponding to an input based on a plurality of batch normalization layers, a plurality of rectified linear unit layers, and a plurality of convolution layers; and
adding the first output and the input to generate a second output, and taking the second output as an output of the second submodel.

10. The prediction model training method of claim 6, wherein the third submodel is configured to perform the following steps:
generating a first output corresponding to an input based on a plurality of first batch normalization layers, a plurality of rectified linear unit layers, and a plurality of first convolution layers;
generating a second output corresponding to an input based on a second convolution layer, a second batch normalization layer, and a max pooling layer; and
adding the first output and the second output to generate a third output, and taking the third output as an output of the third submodel.

11. A prediction model training device, comprising:
a storage, configured to store a first model and a second model; and
a processor, coupled to the storage, wherein the processor is configured to:
training the first model according to a plurality of first electrocardiograms, wherein the first model comprises a feature extraction layer, and the feature extraction layer is configured to extract a plurality of features corresponding to an electrocardiogram;
extracting first feature information corresponding to a plurality of second electrocardiograms according to the plurality of second electrocardiograms and the feature extraction layer of the first model, wherein each of the plurality of second electrocardiograms is corresponding to at least one of the first feature information; and
training the second model according to the first feature information, gender information corresponding to the plurality of second electrocardiograms, and age information corresponding to the plurality of second electrocardiograms.

12. The prediction model training device of claim 11, wherein the processor is further configured to:
receiving a patient electrocardiogram, patient gender information, and patient age information corresponding to a patient;
extracting patient feature information corresponding to the patient electrocardiogram based on the feature extraction layer; and
inputting the patient feature information, the patient gender information, and the patient age information into the second model to generate a prediction result;
wherein the prediction result is configured to indicate whether the patient corresponding to the patient electrocardiogram has a symptom of left ventricular hypertrophy.

13. The prediction model training device of claim 11, wherein the processor is further configured to:
obtaining a plurality of first electrocardiogram segments from each of the plurality of first electrocardiograms, wherein each of the plurality of first electrocardiogram segments corresponds to a time interval in the plurality of first electrocardiograms; and
training the first model according to the plurality of first electrocardiogram segments corresponding to the plurality of first electrocardiograms.

14. The prediction model training device of claim 13, wherein the processor is further configured to:
obtaining a plurality of second electrocardiogram segments from each of the plurality of second electrocardiograms, wherein each of the plurality of second electrocardiogram segments corresponds to the time interval in the plurality of second electrocardiograms; and
extracting the first feature information corresponding to the plurality of second electrocardiograms based on the plurality of second electrocardiogram segments.

15. The prediction model training device of claim 14, wherein each of the plurality of first electrocardiogram segments comprises the time interval corresponding to a peak, and each of the plurality of second electrocardiogram segments comprises the time interval corresponding to the peak.

16. The prediction model training device of claim 11, wherein the first model comprises a first submodel, a second submodel, a third submodel, a fourth submodel, and a fifth submodel, and the processor is further configured to:
training the first model based on a first composition order;
wherein the first composition order corresponds to the fourth submodel, the first submodel, the second submodel, the third submodel, the second submodel, the third submodel, the second submodel, the third submodel, the second submodel, and the fifth submodel.

17. The prediction model training device of claim 11, wherein the second model comprises a fifth submodel, a sixth submodel, and a seventh submodel, and the processor is further configured to:
generating second feature information according to the first feature information and the sixth submodel;
generating a gender vector according to the gender information;
generating an age vector according to the age information; and
training the second model based on the second feature information, the gender vector, the age vector, and a second composition order, wherein the second composition order corresponds to the seventh submodel, the seventh submodel, and the fifth submodel.

18. The prediction model training device of claim 16, wherein the first submodel is configured to perform the following operations:
generating a first output corresponding to an input based on a dropout layer, a plurality of convolution layers, a batch normalization layer, and a rectified linear unit layer;
inputting the input into a max pooling layer to generate a second output; and
adding the first output and the second output to generate a third output, and taking the third output as an output of the first submodel.

19. The prediction model training device of claim 16, wherein the second submodel is configured to perform the following operations:
generating a first output corresponding to an input based on a plurality of batch normalization layers, a plurality of rectified linear unit layers, and a plurality of convolution layers; and
adding the first output and the input to generate a second output, and taking the second output as an output of the second submodel.

20. A prediction model training device, comprising:
a storage, configured to store a first model and a second model; and
a processor, coupled to the storage, wherein the processor is configured to:
training the first model according to a plurality of first electrocardiograms, wherein the first model comprises a feature extraction layer, and the feature extraction layer is configured to extract a plurality of features corresponding to an electrocardiogram;
extracting first feature information corresponding to a plurality of second electrocardiograms according to the plurality of second electrocardiograms and the feature extraction layer of the first model, wherein each of the plurality of second electrocardiograms is corresponding to at least one of the first feature information; and
training the second model according to the first feature information, gender information corresponding to the plurality of second electrocardiograms, and age information corresponding to the plurality of second electrocardiograms, wherein the second model is configured to generate a prediction result according to feature information generated by the first model, the gender information corresponding to the electrocardiogram, and the age information corresponding to the electrocardiogram, and the prediction result is configured to indicate whether a patient corresponding to the electrocardiogram has a symptom of left ventricular hypertrophy.
